(19) United States
(12) Patent Application Publication
(10) Pub. No.: US 2010/0035261 A1
(43) Pub. Date: Feb. 11, 2010

(54) METHOD OF DETECTING LARGE GENOMIC REARRANGEMENTS

(75) Inventors: Thomas Scholl, W. Borough, MA (US); Thaddeus Judkins, Salt Lake City, UT (US); Brant Hendrickson, Shrewsbury, MA (US); Benjamin Roa, Salt Lake City, UT (US); Carrie Colvin, Salt Lake City, UT (US)

Correspondence Address: MYRIAD GENETICS INC.;INTELLECTUAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 12/467,933

(22) Filed: May 18, 2009

Related U.S. Application Data
(63) Continuation of application No. PCT/US2007/085147, filed on Nov. 19, 2007.
(60) Provisional application No. 60/859,681, filed on Nov. 17, 2006.

(51) Int. Cl. C12Q 1/68

(52) U.S. Cl. 435/6

(57) ABSTRACT

A method for detecting large genomic rearrangements is disclosed, which is particularly useful in detecting deletions and duplications in the large genes such as BRCA1, BRCA2, MLH1 and MSH2.

### RELATED APPLICATIONS

This application is a continuation of the international application PCT/US2007/085147 filed on Nov. 19, 2007; which claims the benefit of U.S. Provisional Application Ser. No. 60/859,681 filed Nov. 17, 2006, which are incorporated herein by reference in their entirety.

### FIELD OF THE INVENTION

The invention generally relates to genetic testing, and particularly to method for detecting large genomic rearrangements.

### BACKGROUND OF THE INVENTION

The BRCA1 and BRCA2 genes are tumor suppressor genes identified on the basis of their genetic linkage to familial breast cancers. Mutations in the BRCA1 and BRCA2 genes in humans are associated with predisposition to breast and ovarian cancers. In fact, BRCA1 and BRCA2 mutations are responsible for the majority of familial breast cancer. Inherited mutations in the BRCA1 and BRCA2 genes account for approximately 7-10% of all breast and ovarian cancers. Women with BRCA mutations have a lifetime risk of breast cancer between 56-87%, and a lifetime risk of ovarian cancer between 27-44%.

Human MLH1 and MSH2 genes encode for proteins involved in DNA mismatch repair. Mutations in such DNA mismatch repair genes have been linked to elevated risk of developing various cancers, and may account for up to 90% of the cases of hereditary nonpolyposis colon cancer (HNPCC). HNPCC patients have about 80% increased risk of colon cancer, and elevated risk for cancers of the endometrium, ovary, stomach, small intestine and upper urinary track.

Genetic tests such as BRACAnalysis® and Colaris® have been employed to detect mutations in such cancer predisposition genes in high risk individuals. To date, a large number of deleterious mutations in the BRCA1, BRCA2, MLH1, and MSH2 genes have been discovered. The majority of the mutations are point mutations detectable by DNA sequencing. However, a small percentage of the deleterious mutations are large rearrangements (large deletions or duplications) that are not typically detectable by conventional DNA sequencing.

Southern blot is a common and routine technique for detecting large rearrangement mutations. However, it is not easy to adapt Southern blot to high-throughput clinical lab settings. Hogervorst et al., Cancer Res., 63(7):1449-53 (2003) discloses the so called multiplex ligation-dependent probe amplification (MLPA) technique, a quantitative multiplex ligation and PCR approach to determine the relative copy number of each exon of the genes studied. MLPA uses probes designed to hybridize adjacently to the target sequence. After ligation, the joined probes are amplified and quantified. See also, Gille et al., Br. J. Cancer, 87(8):892-7 (2002). While MLPA is amenable to high throughput, it requires oligonucleotide probes with very long tail sequences especially for complex genes such as BRCA1, BRCA2 and DNA mismatch repair genes. The sensitivity may also need some improvement.

Thus, there is still a need for an improved testing method for detecting large genomic rearrangements useful in clinical testing.

### BRIEF SUMMARY OF THE INVENTION

The present invention provides a sensitive quantitative multiplex endpoint PCR assay designed to detect large arrangements. The method for detecting large genomic rearrangements in one or more genes of a human subject comprises the steps of:

performing a first multiplex PCR to produce a first plurality of amplicons from a plurality of exons or regions of interest of said one or more genes, wherein said first plurality of amplicons do not include any overlapping amplicons;

performing a second multiplex PCR to produce a second plurality of amplicons from said plurality of exons or regions of interest of said one or more genes, wherein said second plurality of amplicons are not identical to said first plurality of amplicons and do not include any overlapping amplicons;

performing a third multiplex PCR to produce said first plurality of amplicons, or a third plurality of amplicons from said plurality of exons or regions of interest of said one or more genes;

optionally performing a fourth multiplex PCR to produce said second plurality of amplicons, or a fourth plurality of amplicons from said plurality of exons or regions of interest of said one or more genes;

wherein said first, second, third and fourth multiplex PCRs are terminated at the exponential phase, e.g., after less than 30 cycles, or between 20 to 30 cycles;

separating said first, second, and third and fourth if present, plurality of amplicons based on size difference; and

analyzing the relative amount of each amplicon produced, whereby detecting the presence or absence of a large genomic rearrangement.

In one embodiment of the method, amplicons generated in the method when combined, comprise substantially all exon sequences of a target gene being interrogated.

In one embodiment of the method, once a deletion of one or more exons is detected in the analyzing step, DNA sequencing is performed on a genomic DNA isolated from the human subject at the region corresponding to the amplicon of the 5′ end of the deletion, and optionally also at the region corresponding to 3′ end of the deletion. This sequencing step is employed to determine the presence or absence of a mutation in the region of the genomic DNA of the human subject where a PCR primer hybridizes.

In another embodiment, the method is used to detect large rearrangements in the human BRCA1 and BRCA2 genes. In yet another embodiment, the method is used to detect large rearrangements in the human MLH1 and MSH2 genes.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 are examples of electropherograms for 9 multiplexes used in the CART assay for large rearrangements in the MLH1 and MSH2 genes, with the high peaks representing the amplicons for MLH1 and MSH2, and the heights corresponding with dosage;

FIG. 2 are electropherograms from one of 9 multiplexes used to provide dosage data at each exon, with the peaks having low amplitudes (black arrows) on the rearrangement-positive samples reflect those exons where only one genomic copy is present;

FIG. 3 is a representative scatter plot data taken from one CART batch of 32 patients;

FIG. 4A is a scatter plot arrayed based on 32 samples processed by Multiplex Ligation-dependent Probe Amplification; and

FIG. 4B is a scatter plot arrayed based on 32 samples processed by CART test.

### DETAILED DESCRIPTION OF THE INVENTION

The present invention provides a process for detecting large genomic rearrangements in one or more genes in a diploid subject, particularly human genes such as human cancer genes that are not on the sex chromosomes.

As used herein, the term “diploid subject” means any diploid biological organisms including, but not limited to, fruit flies, mice, rats, dogs, cats, sheep, cattle, monkeys, and humans.

The term “allele” is used herein to refer generally to one copy of a naturally occurring gene or a particular chromosome region in a diploid subject. A diploid subject has two sets of chromosomes and two copies of a particular gene, and thus two haplotypes of any region of the chromosome and two alleles of any polymorphic site within the gene or chromosome region.

As used herein, the term “genomic rearrangement” means a physical change in a chromosome DNA of a diploid subject that results in an increase or decrease of the copy number of a particular chromosome DNA region, e.g., genomic deletions and duplications.

The term “large genomic rearrangement” refers to genomic deletions and duplications of the entirety of a gene or a portion thereof of a size of at least 50 base pairs.

Generally speaking, the method for detecting large genomic rearrangements in accordance with the present invention comprises the steps of:

(1) providing a sample from a human subject;

(2) performing a first multiplex PCR based on the sample to produce a first plurality of amplicons from a plurality of exons or regions of interest (e.g., promoter region) of one or more genes each comprising a nucleotide sequence of one of said plurality of exons or regions, wherein the first plurality of amplicons do not include any overlapping amplicons;

(3) performing a second multiplex PCR based on the sample to produce a second plurality of amplicons from said plurality of exons or regions each comprising a portion of one of said exons or regions, wherein the second plurality of amplicons are not identical to the first plurality of amplicons and do not include any overlapping amplicons;

performing a third multiplex PCR to produce the first plurality of amplicons, or a third plurality of amplicons from said plurality of exons or regions, and optionally performing a fourth multiplex PCR to produce the second plurality of amplicons, or a fourth plurality of amplicons from said plurality of exons or regions, wherein the first, second, third and fourth multiplex PCRs are terminated at the exponential phase (less than 30 cycles, 20 to 30 cycles);

separating the first, second, and third and fourth if present, plurality of amplicons based on size differences; and analyzing the relative amount of each amplicon produced, whereby detecting the presence or absence of a large genomic rearrangement.

Preferably the first, second, and third and fourth if present, multiplex PCRs are performed simultaneously in one batch.

Also preferably, the amplicons generated in the method together, comprise substantially all exon sequences of a target gene being interrogated.

The method of the present invention is particularly useful in detecting large genomic rearrangements in diploid subjects that are typically not easily detectable by traditional genomic DNA sequencing methods using PCR amplified genomic DNA. For example, such large genomic rearrangements may involve deletions or duplications of one or more full exons of a gene. For example such large genomic rearrangements may involve deletions or duplications of contiguous 50 base pairs, 100 base pairs, 500 base pairs, 1000 base pairs, 2000 base pairs, or 5000 base pairs or more.

In some embodiments, the method of the present invention is to detect large genomic rearrangements in one or more genes chosen from BRCA1, BRCA2, MLH1, MSH2, MSH6, APC, MYH, and other DNA repair genes.

In specific embodiments, the method of the present invention is applied to detect large genomic rearrangements in the BRCA1 and BRCA2 genes. In accordance with such embodiments, the method includes at least the steps of:

(1) providing a genomic DNA containing the BRCA1 and BRCA2 genes from a human subject;

(2) performing a first plurality of multiplex PCRs (preferably using the same amount of the genomic DNA, and more preferably no more than 25 ng in each multiplex PCR), wherein a plurality of test amplicons and at least a control amplicon are produced in each of the first plurality of multiplex PCRs, each of said test amplicons comprises a nucleotide sequence of an exon or promoter region of the BRCA1 or BRCA2 gene, and said plurality of test amplicons in any one multiplex do not include any overlapping amplicons, wherein at least two different amplicons are produced in the first plurality of multiplex PCR from each of the promoter regions and exons of the BRCA1 and BRCA2 genes. That is, at least two different amplicons are produced both comprising a nucleotide sequence of the same exon or promoter region of the BRCA1 or BRCA2 gene. In other words, at least two amplicons having different nucleotide sequences are amplified in the first plurality of multiplex PCRs each of the two amplicons containing a portion of the same exon or promoter region.

In some embodiments, the first plurality of multiplex PCRs includes at least 5, 7, 8 10, or at least 11 or 12 multiplex PCRs, each producing at least 5, 8, 10 or 12 amplicons. In some embodiments, a single multiplex PCR does not produce two test amplicons derived from two adjacent exons, or from a promoter region and the adjacent exon of the BRCA1 or BRCA2 gene.

In some embodiments, all of the test amplicons together in the first plurality of multiplex PCRs comprise substantially all nucleotide sequences of the exons of the BRCA1 and BRCA2 genes.

(3) repeating the step (2) above. The step (3) can be performed separately from step (2) or concurrently in the same batch. The compositions of the multiplex PCRs and the amplicons produced in step (3) can be identical or different from those in step (2). All multiplex PCRs are terminated at the exponential phase, e.g., after no more than 30 cycles, or after 25 to 30 cycles;

(4) after an optional step of purification of the PCR products, separating the amplicons in each multiplex PCR by a capillary sequencer to obtain electropherograms having peaks corresponding the test and control amplicons;

(5) analyzing the electropherograms to deduce the relative amount of each amplicon produced, whereby detecting the presence or absence of a large genomic rearrangement; and optionally

(6) if a deletion of a genomic DNA region is discovered, then at least the portions of the target genomic DNA region where the PCR primers hybridize are independently sequenced to determine if the primer target sequences are identical to the primer sequences. This will eliminate possible false positives caused by the inability of a primer to hybridize to the PCR template thereby causing PCR failure.

Various modifications of such specific embodiments based on the general disclosure of the method of the present invention can be made as will be clear to a skilled artisan.

In other specific embodiments, the method of the present invention is applied to detect large genomic rearrangements in the MLH1 and MSH2 genes. In accordance with such embodiments, the method includes at least the steps of:

(1) providing a genomic DNA containing the MLH1 and MSH2 genes from a human subject;

(2) performing a first plurality of multiplex PCRs (preferably using the same amount of the genomic DNA, and more preferably no more than 25 ng in each multiplex PCR), wherein a plurality of test amplicons and at least a control amplicon are produced in each of the first plurality of multiplex PCRs, each of said test amplicons comprises a nucleotide sequence of an exon or promoter region of the MLH1 or MSH2 gene, and said plurality of test amplicons in any one multiplex do not include any overlapping amplicons, wherein at least two different amplicons are produced in the first plurality of multiplex PCR from each of the promoter regions and exons of the MLH1 and MSH2 genes. That is, at least two different amplicons are produced both comprising a nucleotide sequence of the same exon or promoter region of the MLH1 or MSH2 gene. In other words, at least two amplicons having different nucleotide sequences are amplified in the first plurality of multiplex PCRs each of the two amplicons containing a portion of the same exon or promoter region.

In some embodiments, the first plurality of multiplex PCRs includes at least 5, 7, 8 10, or at least 11 or 12 multiplex PCRs, each producing at least 5, 8, 10 or 12 amplicons. In some embodiments, a single multiplex PCR does not produce two test amplicons derived from two adjacent exons, or from a promoter region and the adjacent exon of the MLH1 or MSH2 gene.

In some embodiments, all of the test amplicons together in the first plurality of multiplex PCRs comprise substantially all nucleotide sequences of the exons of the MLH1 and MSH2 genes.

(3) repeating the step (2) above. The step (3) can be performed separately from step (2) or concurrently in the same batch. The compositions of the multiplex PCRs and the amplicons produced in step (3) can be identical or different from those in step (2). All multiplex PCRs are terminated at the exponential phase, e.g., after no more than 30 cycles, or after 25 to 30 cycles;

(4) after an optional step of purification of the PCR products, separating the amplicons in each multiplex PCR by a capillary sequencer to obtain electropherograms having peaks corresponding the test and control amplicons;

(5) analyzing the electropherograms to deduce the relative amount of each amplicon produced, whereby detecting the presence or absence of a large genomic rearrangement; and optionally

(6) if a deletion of a genomic DNA region is discovered, then at least the portions of the target genomic DNA region where the PCR primers hybridize are independently sequenced to determine if the primer target sequences are identical to the primer sequences. This will eliminate possible false positives caused by the inability of a primer to hybridize to the PCR template thereby causing PCR failure.

Various modifications of such specific embodiments based on the general disclosure of the method of the present invention can be made as will be clear to a skilled artisan.

Typically, in the method of the present invention, a sample is obtained from a diploid subject to be tested. The sample can be a tissue specimen such as blood or buccal swab or any other specimens having one or more cells containing genomic DNA. The sample can also be genomic DNA extracted from a tissue specimen. A quantitative multiplex PCR endpoint assay is then performed using the sample obtained from the diploid subject. PCR amplification can be performed directly using a tissue specimen or using extracted genomic DNA. Preferably, for all multiplex PCRs in a batch performed in the same time, the same amount of genomic DNA is used as template in each multiplex PCR. In preferred embodiment, less than 25 nanograms of total genomic DNA is used in each multiplex PCR.

In the quantitative multiplex endpoint PCR assay, a plurality of quantitative multiplex PCR reactions is performed. Preferably each multiplex PCR produces at least 5 amplicons. The number of multiplexes is variable and is determined by the total number of amplicons to be produced. In some embodiments, multiplex PCR amplifications are performed wherein each multiplex PCR reaction amplifies at least 5, 6, 7, 8, 9, 10, or 12 different regions (e.g., exons). Within each multiplex, the sizes of the amplicons are sufficiently different so that the amplicons are distinguishable and identifiable once separated by size differences by, e.g., electrophoresis, in a polyacrylamide gel, agarose gel or capillary sequencer. Typically, the amplicons have a size (the length of each amplified DNA fragment) from about 40 base pairs to about 1000 base pairs, preferably from about 50 or 100 to about 500 base pairs. The amplicons can be generated by amplifying a region of the template genomic DNA. This is the region being examined by the method of the present invention to determine whether the region is deleted or duplicated in one or both alleles. Such a region can be a promoter region, an intronic sequence, an exonic sequence, or have both an intronic sequence and an exonic sequence. In one preferred embodiment, each amplicon contains a portion of an exon or a promoter of a gene being examined. That is, while the PCR primers can hybridize to intron sequences or exon sequences or both, each pair of reverse and forward primers must be designed to amplify a portion of an exon or a promoter sequence. In some embodiments, a multiplex PCR is performed to amplify a plurality of regions of one or more genes to be detected. Such regions can be exonic or intronic or a hybrid region having both exonic and intronic sequences, or a promoter sequence. In preferred embodiments, a multiplex PCR is performed to amplify all exons of one or more genes to be detected.

In the method of the present invention, each multiplex does not contain two overlapping amplicons. By “overlapping amplicons” it is referred to two amplicons that are generated from two overlapping regions of the same genomic DNA template. In addition, preferably each genomic region being examined is represented by at least two amplicons that preferably overlap each other, but are not identical. Such two amplicons must be in separate multiplexes.

In preferred embodiments of the present invention, amplicons produced with adjacent exons as templates are not included in the same multiplex PCR. That is, a plurality of amplicons generated in one multiplex PCR do not include such two amplicons one of which comprises a portion or the entirety of the sequence of a first exon and the other amplicon comprises a portion or the entirety of the sequence of an exon that is adjacent to (i.e., separated only one intron from) the first exon. In this manner, multiexonic rearrangements (large rearrangements involving multiple exons) are identified in a more independent manner.

In addition, preferably each multiplex also contains one or more control amplicons for normalization purposes in quantitative analysis discussed below. Control amplicons are produced from the same sample of a diploid subject, but using one or more pairs of primers each flanking a region (e.g., a whole or portion of an exon) of a housekeeping gene. As used herein, a “housekeeping” gene means a gene that is almost always present in two copies in such cells from a living and normal diploid subject and neither copy harbors a genomic rearrangement. Examples of such housekeeping genes are well known in the art and would be apparent to a skilled artisan. Examples of suitable housekeeping genes include, but are not limited to the GAPDH and β-actin genes. For purposes of clarity, “test amplicon” is used herein in contrast to “control amplicon,” and refers to an amplicon produced using a genomic DNA of a gene being examined for the presence or absence of a large rearrangement therein.

In preferred embodiments, each region to be detected is amplified by PCR with a first primer pair including a first primer and a second primer hybridizing under PCR conditions to a 5′ and a 3′ end sequence flanking the region, respectively, and in a separate PCR reaction with a second pair of primers including a third primer and a fourth primer hybridizing under PCR conditions to sequences 5′ to and 3′ to and flanking the region, respectively, wherein the first and second pairs are not identical, and preferably, the first and third primers, and the second and fourth primers, do not both overlap (the first and third primers overlap each other, but the second and fourth primers do not, or vice versa, or neither the first and third or the second and fourth overlap). In these preferred embodiments, also preferably at least one region (e.g., exon) of a housekeeping gene is amplified to produce a control amplicon.

In preferred embodiments, at least one first PCR primer pair is designed such that each comprises two contiguous portions: (1) a first primer having a first 5′ portion having from about 15 to about 25 nucleotides (preferably from about 18 to about 20 nucleotides), and a first 3′ portion having a contiguous span of from about 15 to about 40 (preferably from about 18 to about 36) nucleotides of the target genomic DNA sequence to be amplified or the complement thereof, and sufficient to enable hybridization of the primer to the 5′ end of the target genomic DNA region under ordinary PCR annealing conditions; and (2) a second primer having a second 5′ portion having from about 15 to about 25 nucleotides (preferably from about 18 to about 20 nucleotides), and a second 3′ portion having a contiguous span of from about 15 to about 40 (preferably from about 18 to about 36) nucleotides of the target genomic DNA sequence to be amplified or the complement thereof, and sufficient to enable hybridization of the primer to the 3′ end of the target genomic DNA region under ordinary PCR annealing conditions. Typically, a sample from a diploid subject such as human sample is divided into at least two portions. One portion of the sample is used for PCR reactions for the amplification of the target regions using at least the first primer pair described above. In addition, a separate contamination control PCR reaction is performed on a second portion of the same sample using a control primer pair: a first control primer having a sequence substantially identical to the first 5′ portion of the above-described first primer such that it is capable of hybridizing to the first 5′ portion of the above-described first primer, but not to the first 3′ portion of the above-described first primer; and a second control primer having a sequence substantially identical to the second 5′ portion of the above-described second primer such that it is capable of hybridizing to the second 5′ portion of the above-described second primer, but not to the second 3′ portion of the above-described second primer.

One multiplex reaction is included to check for PCR product contamination in the laboratory setting. If a PCR product is produced in the contamination control PCR reaction, then the entire result from the amplification of the target regions is discarded and disregarded.

In preferred embodiments, all samples are run in duplicate within a batch. For example each multiplex reaction is performed in duplicates. Alternatively, more than two amplicons, e.g., 3, 4 or 5 are amplified separately in different multiplex PCR from each region (e.g., an exon or a promoter region) of a genomic DNA template. The multiple amplicons from the same region can be identical or different (e.g., varying in length or sequence). This way, multiple data points are generated for each genomic DNA region, and the power of statistical analysis is increased.

The quantitative multiplex PCR amplifications are conducted in such a manner that the PCR reactions are stopped at the exponential phase of the reaction, that is, when the number of amplified molecules (if present) of each amplicon is in linear proportional relationship to the number of PCR amplification cycle. In this manner, if the diploid subject has a deletion in a region to be amplified, then the total number of amplified DNA molecules in that region (test amplicon) will be about a half of that of another amplicon from another region where the diploid subject has two copies. For example, the multiplex PCRs can be terminated after less than 30 cycles, preferably between 20 to 30 cycles.

Preferably the amplicons are labeled with a detectable marker for easy detection and quantification of the amount of each amplicon. For example, PCR primers can be labeled with fluorescence or radioactive isotope or the like. Alternatively, the amplicons can be labeled during PCR reaction by incorporation of labeled nucleotides into the amplicons.

After multiplex PCR, optionally the PCR products are purified to remove residual primers and/or deoxyribonucleotides. The amplicons are separated based on size differences, e.g., by electrophoresis, in a polyacrylamide gel, agarose gel or capillary sequencer, and each amplified DNA fragment (amplicon) is quantified to determine the amount of DNA produced in each amplicon. In some embodiments, an internal size standard is included during the size-based separation. For example, a plurality of DNA fragments of known but varying sizes can be mixed with each multiplex PCR product mix (which contains a plurality of amplicons), and are run in electrophoresis or capillary sequencer. The sizes and identities of each amplicon produced in a multiplex PCR can be established by comparison with the size standard.

To determine the copy number of each region of a genomic DNA (or gene) examined, the amount of each test amplicon is determined and compared to or normalized against one or more other test amplicons (e.g., amplicons amplified from one or more different exons in the same gene and/or one or more different genes) and/or the amount of one or more control amplicons. The amount of each amplicon can be determined, for example, by measuring the height of a peak corresponding to the amplicon in an electropherogram after the multiplex PCR products are run on a capillary sequencer. In one embodiment, since each control amplicon from the housekeeping genes has two copies (i.e. no deletion or duplication), any duplication or deletion, i.e., copy number change in the region from which a test amplicon is amplified would be detected by the comparison or normalization to determine the relative copy number of the test amplicon. For example, if the copy number of a test amplicon is determined to be about half of that of a control amplicon, this would indicate a deletion in one allele.

The amount measurement and copy number analysis can be done manually by a human subject, or by computer. In one embodiment, the separation of amplicons within each multiplex is accomplished by a capillary sequencer, and a electropherogram is obtained. In one embodiment, the amount measurement and copy number analysis entail the steps of: (1) determine the amplitude (height) of each peak corresponding to each amplicon; (2) obtain a “first gene median peak height” which is the median of the peak heights of all test amplicons in each multiplex from a first gene and control amplicon(s) in the same multiplex, and normalize each test amplicon from a second gene in the same multiplex against the “first gene median peak height” to obtain a plurality of “normalized test amplicon peak height” corresponding to the test amplicons from the second gene in the multiplex; (3) obtain a “second gene median peak height” which is the median of the peak heights of all test amplicons in each multiplex from a second gene and control amplicon(s) in the same multiplex, and normalize each test amplicon from the first gene in the same multiplex against the “second gene median peak height” to obtain a plurality of “normalized test amplicon peak height” corresponding to the test amplicons from the first gene in the multiplex; (4) obtain a “median normalized peak height value” which is the median of all “normalized test amplicon peak height” for a particular amplicon in different patients tested in the same batch, and normalize the “normalized test amplicon peak height” for each amplicon against the thus obtained “median normalized peak height value” to arrive at a “secondary normalized test amplicon peak height” for each amplicon; (4) obtain a “normalized exon peak height” by averaging all “secondary normalized test amplicon peak height” for all amplicons derived from a particular exon; and optionally (5) plot the “normalized exon peak height” for each exon on a scatter plot.

The above analysis steps can be done manually or by computer means. For example, the analysis steps can be implemented using hardware, software or a combination thereof in one or more computer systems or other processing systems capable of effecting the steps described above within the system. The computer-based analysis function can be implemented in any suitable language and/or browsers. For example, it may be implemented with C language and preferably using object-oriented high-level programming languages such as Visual Basic, SmallTalk, C++, and the like. The application can be written to suit environments such as the Microsoft Windows™ environment including Windows™ 98, Windows™ 2000, Windows™ NT, and the like. In addition, the application can also be written for the MacIntosh™, SUN™, UNIX or LINUX environment. In addition, the functional steps can also be implemented using a universal or platform-independent programming language. Examples of such multi-platform programming languages include, but are not limited to, hypertext markup language (HTML), JAVA™, JavaScript™, Flash programming language, common gateway interface/structured query language (CGI/SQL), practical extraction report language (PERL), AppleScript™ and other system script languages, programming language/structured query language (PL/SQL), and the like. Java™-or JavaScript™-enabled browsers such as HotJava™, Microsoft™ Explorer™, or Netscape™ can be used. When active content web pages are used, they may include Java™ applets or ActiveX™ controls or other active content technologies.

A useful computer system for implementing the analysis functions described above may comprise an interface module for receiving data of the amount and/or identity of each amplicon in the plurality of multiplex PCRs; and one or more computer program means for performing the analysis steps described above.

The analysis function can also be embodied in computer program products and used in the systems described above or other computer- or internet-based systems. Accordingly, another aspect of the present invention relates to a computer program product comprising a computer-usable medium having computer-readable program codes or instructions embodied thereon for enabling a processor to carry out the analysis steps described above. These computer program instructions may be loaded onto a computer or other programmable apparatus to produce a machine, such that the instructions which execute on the computer or other programmable apparatus create means for implementing the functions or steps described above. These computer program instructions may also be stored in a computer-readable memory or medium that can direct a computer or other programmable apparatus to function in a particular manner, such that the instructions stored in the computer-readable memory or medium produce an article of manufacture including instruction means which implement the analysis functions or steps. The computer program instructions may also be loaded onto a computer or other programmable apparatus to cause a series of operational steps to be performed on the computer or other programmable apparatus to produce a computer implemented process such that the instructions which execute on the computer or other programmable apparatus provide steps for implementing the functions or steps described above.

In some embodiments, if a deletion of a genomic DNA region is discovered, e.g., based on a reduction of copy number of the target region to one or zero, then at least the portions of the target genomic DNA region where the PCR primers hybridize are independently sequenced to determine if the primer target sequences are identical to the primer sequences. This will eliminate possible false positives caused by the inability of a primer to hybridize to the PCR template thereby causing PCR failure.

Various modifications of such specific embodiments based on the general disclosure of the method of the present invention can be made as will be clear to a skilled artisan.

### Example 1

Hereditary non-polyposis colon cancer (HNPCC) is caused by germline mutations in the mismatch repair genes MLH1, MSH2, MSH6 and PMS2. HNPCC patients have ˜80% increased risk of colon cancer, and elevated risk for cancers of the endometrium, ovary, stomach, small intestine and upper urinary tract. Molecular genetic testing in HNPCC families showed that ˜90% of cases are attributed to MLH1 and MSH2, 7-10% to MSH6, and <5% to PMS2. The majority are point mutations detectable by sequencing; however, approximately 5% and 20% of mutations in MLH1 and MSH2, respectively, are large rearrangements that require other detection techniques such as Southern blot or multiplex ligation-dependent probe amplification (MLPA™). Our laboratory had previously developed and implemented a quantitative multiplex PCR (QMPCR) endpoint assay for clinical testing for large rearrangements in the BRCA1 and BRCA2 genes. We have developed a similar assay for the MLH1 and MSH2 genes in HNPCC which we refer to as CART (COLARIS® Rearrangement Test). The CART test consists of 9 multiplexes of 8-12 amplicons each, with at least 2 amplicons targeting each coding exon, promoter, and 3′UTR of both genes. Copy numbers are normalized against MLH1, MSH2, and two unlinked control genes. Internally developed software provides automated analysis and statistical confidence levels for the presence or absence of large rearrangements. Initial validation of CART has been performed on 14 MLH1 or MSH2 rearrangement-positive and 30 negative DNA samples. Results were 100% concordant with previous data on routine Southern blots, as well as supplemental MLPA™studies. CART has greatly improved turnaround time, accuracy, and consistency compared to Southerns and MLPA™. QMPCR is a superior diagnostic tool for detecting large rearrangements in disease genes. Validation of CART for MLH1 and MSH2 rearrangements is underway on a larger set of previously genotyped samples in a blinded manner. In conjunction with sequencing of the MLH1 and MSH2 genes, the CART assay is expected to improve molecular diagnostic testing on individuals at risk for HNPCC.

The mutation profile of the MLH1 and MSH2 genes associated with HNPCC consists of point mutations and small rearrangements involving a few bases, as well as large deletions and duplications that are refractory to sequencing analysis. Since approximately 5% of MLH1 mutations and ˜20% of MSH2 mutations are large rearrangements, additional methods are required to maximize the sensitivity of HNPCC testing. Our laboratory had performed sequencing of the MLH1 and MSH2 genes plus Southern blot analysis on patients referred for comprehensive HNPCC testing (Comprehensive COLARIS®). Negative patient samples have the option of reflex testing by MSH6 sequencing, which detects mutations in ˜2% of HNPCC cases. Southern analysis is highly effective; however, it has several drawbacks that include: a) requirement for large amounts of high molecular weight DNA, b) technical challenges in routine testing, and c) long turnaround time. To improve on these limitations, we developed and validated a quantitative multiplex PCR endpoint assay for large rearrangements in the MLH1 and MSH2 genes that we have designated as the COLARIS® Rearrangement Test (CART). We present our results from our expanded clinical validation studies, and show data demonstrating superior performance of CART compared to multiplex ligation-dependent probe amplification (MLPA™) kit for MLH1 and MSH2 large rearrangement analysis.

The COLARIS® Rearrangement Test (CART) is a quantitative multiplex PCR endpoint assay designed to detect large rearrangements in MLH1 and MSH2. This dosage-sensitive PCR assay was developed to replace Southern analysis for deletions and duplications, which our laboratory performed in conjunction with MLH1 and MSH2 full gene sequencing on HNPCC patients referred for comprehensive COLARIS® testing. The CART assay consists of 9 PCR multiplexes with a depth of between 8 and 12 amplicons per multiplex, as well as one multiplex for contamination detection. Each coding exon, promoter and 3′UTR of both MLH1 and MSH2 are represented by at least two amplicons that are located in separate multiplexes. These multiplexes also contain control amplicons from two unlinked genes for normalization purposes. Furthermore, target amplicons for contiguous exons are not placed in the same multiplex so that multiexonic rearrangements are identified in a more independent manner. PCR chemistry of certain multiplexes was optimized to enhance amplification of GC-rich regions. Fluorescently labeled PCR primers were designed to avoid common SNPs that could alter primer annealing and amplification. The PCR thermocycling reaction is terminated in the linear phase, and purified PCR products are fractionated by capillary electrophoresis. Relative copy numbers of target PCR products are analyzed using an internally-developed software application. Corresponding peak heights for MLH1 are normalized against MSH2, and vice versa, as well as the control genes, to determine rearrangements with statistical confidence values.

The results from the analysis of the peak heights are arrayed in a scatter plot to easily view the samples and their rearrangements. Individual MLH1 and MSH2 gene regions are represented by different points on the x-axis of the scatter plot; a normal 2× copy number are shown by data symbols clustered around the midpoint on the y-axis. Deleted exons in MLH1 and MSH2 are represented by data points at a 0.5 to 1 relative ratio, and duplications are represented by data points at a 1.5 to 1 ratio. All samples are run in duplicate within a batch, and batches contain 31 patient samples and a positive control. Putative rearrangement-positive samples are run through the assay a second time for confirmation. In addition, sequencing is performed on deletion-positive samples to ensure that no rare polymorphisms underlie CART multiplex primers. Without this quality control measure, primer binding SNPs could yield false-positive results for PCR dosage-sensitive assays.

The purpose of the expanded validation study is to perform the CART assay on a large number of samples previously tested by Southern blot to confirm that CART can detect the same rearrangements in exonic regions with 100% confidence. The clinical validation study has tested 516 patient samples, including 116 known positive and 400 known negative patient samples, by CART in a blinded manor. Each rearrangement-positive sample was confirmed by repeating the CART assay. Long-range PCR was done in some instances to determine the orientation of a putative duplication. Additional sequencing was performed on putative deletion-positive samples to rule out artifacts due to primer binding SNPs. Overall, our results showed that CART was 100% successful in detecting all exonic rearrangements in concordance with previous results from routine testing by Southern blot analysis, as well as supplemental studies by MLPA™ (MRC Holland).

### Comparison of CART, Southern Blot, and MLPA

Currently used methods for diagnostic testing for HNPCC include Southern blot and MLPA analysis. Our Southern blot analysis for MLH1 and MSH2 employed three different restriction enzyme digests. Visual inspection of Southern blot data by multiple reviewers facilitated by Phosphorimager trace analysis detected deletions and duplications in genomic DNA. Southern analysis yielded consistent results that are largely considered the “gold standard” for rearrangement testing. CART provides results consistent with Southern blots with the additional advantages that include: a) reduced requirement for starting amount of patient DNA, b) enhanced laboratory workflow facilitated by automated processes, and c) significantly reduced technical turnaround time of two days instead of one week.

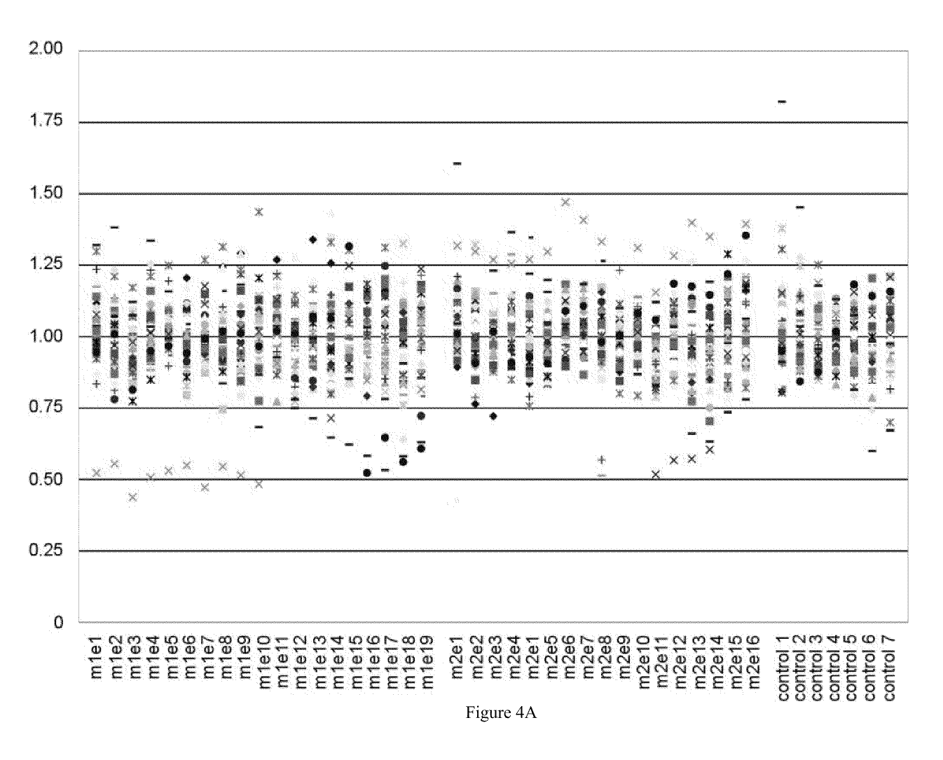

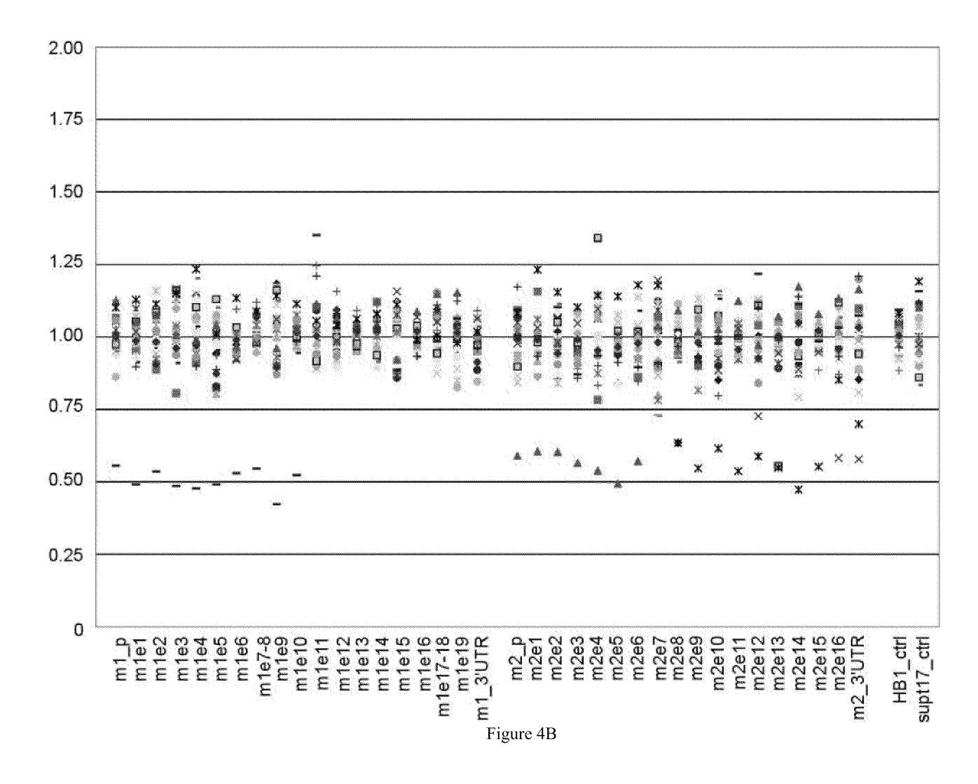

An alternative used in other laboratory settings is the MLPA™ method, which is based on oligonucleotide probe annealing, ligation and PCR amplification. During our validation study, we tested 96 HNPCC samples using CART and MLPA™ methodologies. Results from both assays were calculated as per the CART method and arrayed in scatter plots. To generate these scatter plots, peak heights for the amplicons in both CART and MLPA™ were obtained from the capillary electrophoresis data output from the GeneMarker™ software application (SoftGenetics) and analyzed using a Microsoft Excel macro. Manual analysis in this comparison with CART consisted of normalizing peak heights as outlined in “CART Assay Design” (above). As shown in FIGS. 4A and 4B, careful design of the CART assay, which results in 4 data points per exon (each DNA run in duplicate with 2 amplicons per exon), coupled with a powerful analysis tool, allows for a much tighter distribution of data points and lower coefficients of variation as compared to MLPA™. In addition, rearrangements are detected with higher statistical confidence.

### Conclusions

COLARIS® Rearrangement Test (CART) is a robust and superior clinical test for large rearrangement mutations in MLH1 and MSH2.

An expanded CART assay validation was completed on 116 rearrangement-positive and 400 negative samples. All results were 100% concordant with previous routine tests by Southern blot and supplemental studies by MLPA™.

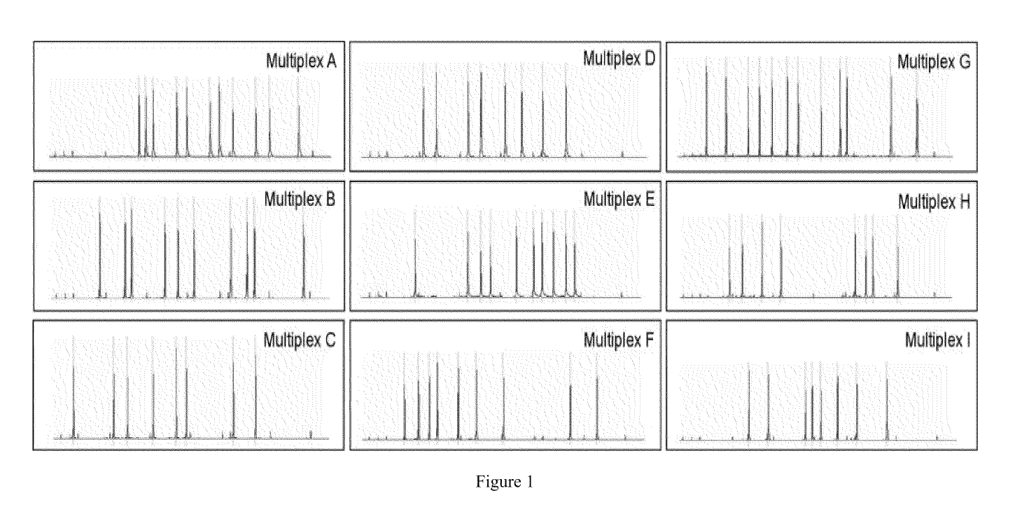

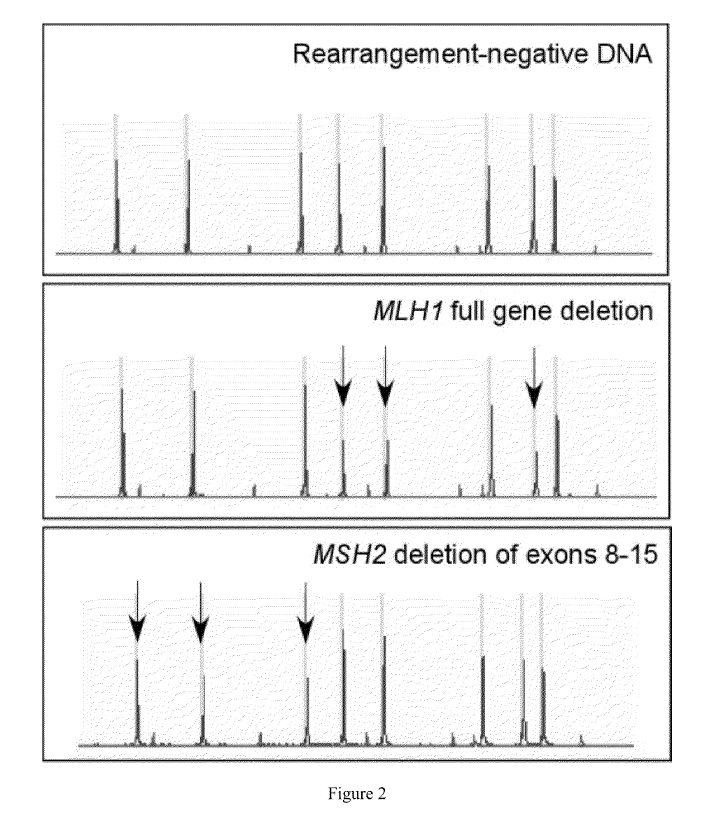

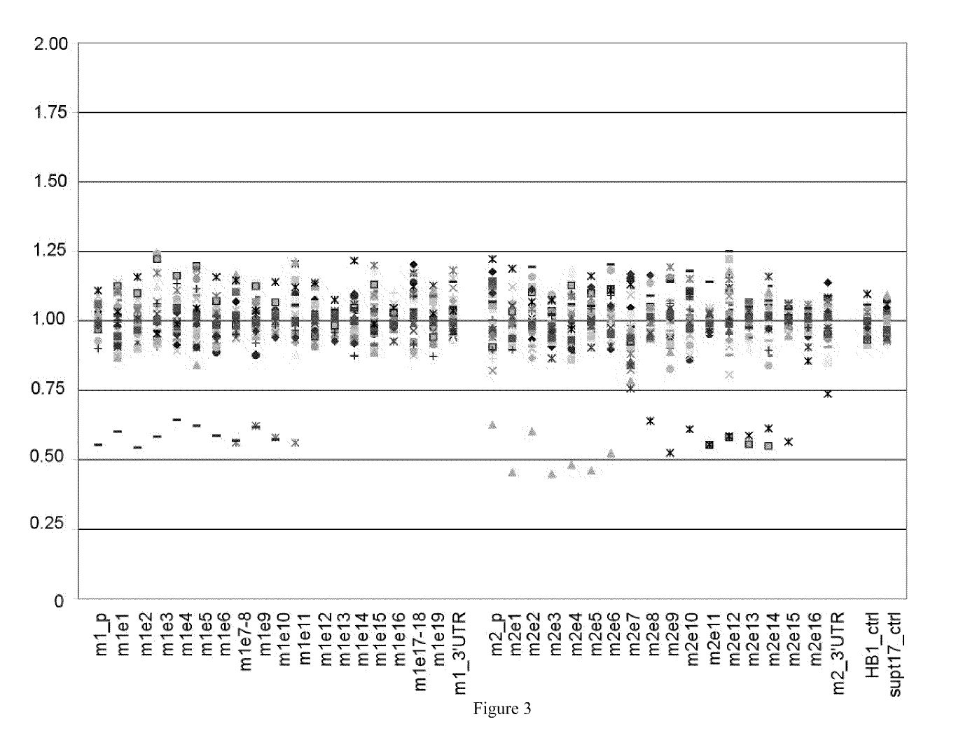

The advantages of CART include: a) reduced requirement for starting amount of patient DNA, b) enhanced laboratory workflow facilitated by automated processes, and c) significantly reduced technical turnaround time. The results are illustrated in FIGS. 1-4.

### Example 2

### 1. BART (BRCA1/2 Rearrangement Test) Assay and Process Features

We used existing and specifically generated BRCA1 and BRCA2 sequence data to avoid common polymorphisms in BART primer design. BART multiplex PCR reactions were designed to interleave BRCA1 and BRCA2 amplicons avoid data artifacts involving contiguous gene regions. BART multiplex PCR reactions were designed to group 2 sets of GC-rich amplicons to optimize reactions using GC-rich PCR chemistry. Relative dosage of individual amplicons was assessed using analytical software tool developed to assess deletion or duplication mutations within BRCA1 and BRCA2. The software provides probability scores for mutation positive calls. BART samples are run in an automated manner with barcode tracking throughout process. Positive samples are re-queued using BART for confirmatory testing.

Samples that test positive for deletion by BART are checked for BRCA1/2 sequences corresponding to the relevant BART primer binding sites. This is to assess possible rare sequence variants that could affect BART primer binding leading to a false positive result for deletion on the dosage-sensitive BART assay.

Mutations in the BRCA1 and BRCA2 genes are comprised of a majority of mutations that are detectable by sequence analysis, and a minority of deletion and duplication mutations that are refractory to sequencing. To provide options for enhanced BRCA1 and BRCA2 molecular test sensitivity, our laboratory developed and implemented a clinical assay for large rearrangements that we refer to as BART (BRCA1/2 Rearrangement Test). We previously validated BART clinically using a large number of breast/ovarian cancer patient samples in a blinded manner. We also demonstrated superior performance of BART versus other dosage-sensitive methods including Multiplex Ligation-dependent Probe Amplification (MLPA).

Methods: BART utilizes quantitative endpoint polymerase chain reaction (PCR) in a multiplexed fluorescent format. Eleven multiplex PCR reactions were designed to contain two amplicons targeting the promoter region, all coding exons, and flanking regions of BRCA1 and BRCA2. An automated likelihood-based analysis application normalizes target amplicon copy number between BRCA1, BRCA2 and three control genes. Deletions and duplications are identified with a statistical confidence level.

Results: Based on clinical and family history criteria, 1,035 patients were identified as high-risk during the initial months of clinical BART analysis at Myriad Genetic Laboratories. All patients were initially tested for Comprehensive BRACAnalysis which includes BRCA1 and BRCA2 full gene sequencing plus large rearrangement panel testing for 5 recurrent BRCA1 mutations. Among high-risk patients, 302 (29.2%) were positive for a BRCA1 or BRCA2 mutation by sequencing and 9 (0.9%) were positive by large rearrangement panel. Patients who tested negative underwent BART reflex testing for unknown deletions or duplications. Twenty seven high risk patients (2.6%) tested positive by BART for various deletions and duplications in BRCA1 and BRCA2. The total mutation detection rate among 1,035 high-risk patients was 32.7%; 11% of mutations were large rearrangements of which 8.0% (27/338) were identified by BART.

Conclusion: Our initial clinical data indicate that BART testing is appropriate for high-risk patients identified on the basis of personal and family history criteria.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

What is claimed is:

1. A method for detecting large genomic rearrangements in one or more genes of a human subject, said method comprising:
providing a sample having genomic DNA of said one or more genes from said human subject;
performing a first multiplex PCR using the sample to produce a first plurality of amplicons each comprising a nucleotide sequence of an exon of said one or more genes, wherein said first plurality of amplicons do not include any overlapping amplicons;
performing a second multiplex PCR to produce a second plurality of amplicons each comprising a portion of an exon of said one or more genes, wherein said second plurality of amplicons are not identical to said first plurality of amplicons and do not include any overlapping amplicons;
performing a third multiplex PCR to produce said first or second plurality of amplicons, or a third plurality of amplicons from said plurality of exons of said one or more genes, wherein said first, second and third multiplex PCRs are terminated at the exponential phase;
separating said first, second, and third if present, plurality of amplicons based on size differences; and
analyzing the relative amount of each amplicon produced, whereby detecting the presence or absence of a large genomic rearrangement.

2. The method of claim 1, wherein each of said first, second and third plurality of amplicons comprises a control amplicon, and said analyzing step comprises comparing the amount of each amplicon to the amount of said control amplicon.

3. The method of claim 1, wherein at least 5 amplicons are produced in each of said first, second and third multiplex PCR.

4. The method of claim 1, wherein none of said first, second and third plurality of amplicons comprises two amplicons having exon sequences from two adjacent exons.

5. The method of claim 1, further comprising sequencing a region of the genomic DNA where a PCR primer used in producing an amplicon hybridizes to, if a large genomic rearrangement is detected based on the decrease of the amount of said amplicon.
